(19) United States
(12) Patent Application Publication
(10) Pub. No.: US 2004/0030186 A1
(43) Pub. Date: Feb. 12, 2004

(54) Process for preparing 3,3-dimethyl-2-oxobutyric acid

(76) Inventor: Helmut Fiege, Leverkusen (DE)

Correspondence Address: BAYER CHEMICALS CORPORATION, PATENT DEPARTMENT, 100 BAYER ROAD, PITTSBURGH, PA, 15205-9741, US

(21) Appl. No.: 10/637,274

(22) Filed: Aug. 8, 2003

(30) Foreign Application Priority Data
Aug. 12, 2002 (DE) 10236919.4

(51) Int. Cl. C07C 59/347

(52) U.S. Cl. 562/577

(57) ABSTRACT

The invention relates to a process for preparing 3,3-dimethyl-2-oxobutyric acid and salts thereof by oxidizing 3,3-dimethyl-2-hydroxybutyric acid with oxygen or oxygen-containing gases in a basic aqueous medium and in the presence of palladium catalysts and also of bismuth or bismuth compounds.

### BACKGROUND OF THE INVENTION

1. Field of the Invention

The invention relates to a process for preparing 3,3-dimethyl-2-oxobutyric acid and salts thereof by oxidizing 3,3-dimethyl-2-hydroxybutyric acid with oxygen or oxygen-containing gases in a basic aqueous medium and in the presence of palladium catalysts and also of bismuth or bismuth compounds.

2. Brief Description of the Prior Art

3,3-Dimethyl-2-oxobutyric acid, especially in the form of its aqueous sodium salt solution, is used for industrial scale preparation of agrochemicals, in particular the selective herbicide, Metribuzin (Sencor®) which is required predominantly in cultures of soya beans, tomatoes and potatoes, (see also U.S. Pat. No. 3,671,523 and U.S. Pat. No. 3,905,801).

Related to the preparation of 3,3-Dimethyl-2-oxybutyric acid is EP-A 1 097 917, which discloses a liquid phase oxidation of 2-hydroxycarboxylic acids or esters thereof using hypobromous acid. However, the use of hypobromous acid as the oxidizing agent on the industrial scale is unacceptable for ecological and economic reasons.

EP-A 0 011 207 discloses a process for preparing 3,3-dimethyl-2-oxo-butyric acid and/or its salts which proceeds via the oxidation of 3,3-dimethyl-2-hydroxybutyric acid in aqueous alkaline solution using hypochlorous acid (hypochlorite) in the presence of a ruthenium catalyst. However, in this case also, the cost and the low environmental compatibility of the oxidizing agent impair industrial application.

A similar process which is described in the U.S. Pat. No. 5,091,568 circumvents these disadvantages by using oxygen as the oxidant. However, distinctly higher catalyst amounts of up to 5% by weight and a high oxygen pressure of 20 to 40 bar are required, which makes the process inefficient.

Also, DE-A 29 15 395 discloses that sodium 2-hydroxypropionate can be oxidized using oxygen or air over a platinum catalyst in the presence of bismuth or lead or compounds thereof at 35-70° C. to give yields of 8-90% of sodium 2-oxopropionate. The application to the oxidation of 3,3-dimethyl-2-hydroxybutyric acid or the sodium salt thereof is not described. However, the process described is completely unsuited thereto (see comparative examples).

There was therefore the need to develop a process which makes it possible to prepare 3,3-dimethyl-2-oxobutyric acid or salts thereof in an efficient manner.

### SUMMARY OF THE INVENTION

A process has now been found for preparing 3,3-dimethyl-2-oxobutyric acid or salts thereof by oxidizing 3,3-dimethyl-2-hydroxybutyric acid with transition metal catalysis, which is characterized in that it is carried out in the presence

of oxygen,

palladium catalyst,

bismuth and/or bismuth compounds and

base.

Within the scope of the invention as described more fully hereunder, all of the parameters and illustrations listed in general or within areas of preference, and thus also the particular areas and areas of preference, may be combined as desired.

### DETAILED DESCRIPTION OF THE INVENTION

The 3,3-dimethyl-2-hydroxybutyric acid in the process according to the invention can be used in the D-form, the L-form or in any desired mixtures of the enantiomers, in particular the racemic form. Also, 3,3-dimethyl-2-hydroxybutyric acid can be used at least partly in the form of a salt, in particular an alkali metal salt.

3,3-Dimethyl-2-hydroxybutyric acid can be prepared by known processes, for example by reacting dichloropinacoline with aqueous alkali (see also DE-A 26 48 300). The resulting alkali metal salt solution of 3,3-dimethyl-2-hydroxybutyric acid can, for example, be used directly for the process according to the invention.

When the process according to the invention is carried out, 3,3-dimethyl-2-oxobutyric acid is obtained predominantly in the form of its salts, from which the free acid can be obtained, if required, by acidification. However, the reaction solution obtained can optionally also be used directly for further reactions, for example with thiocarbohydrazide to form 4-amino-6-tert-butyl-3-thio-1,2,4-triazin-5(4H)-one.

Examples of useful palladium catalysts include metallic palladium, in particular palladium black or palladium applied to a support. Examples of useful supports therefor include activated carbon, graphite, kieselguhr, silica gel, spinels, aluminium oxides, calcium carbonate, magnesium oxide, barium sulphate and also organic support materials.

The supports used are preferably pulverulent activated carbons, for example medicinal carbons or activated carbons produced from wood, as frequently used for decolorizing purposes.

The palladium content of the supported catalysts can be varied within a wide range. In general, supported catalysts are used whose palladium content is between 0.1 and 20% by weight, preferably between 0.5 and 15% by weight.

The amount in which the palladium catalyst is used can also be varied within a relatively wide range. Among other factors, it depends on the desired oxidation rate. In general, the amount of catalyst selected is such that the molar ratio of palladium to 3,3-dimethyl-2-hydroxybutyric acid or salts thereof used is between 1:5 and 1:20,000, preferably between 1:10 and 1:10,000, and particularly preferably between 1:100 and 1:10,000.

The activity and selectivity of the palladium catalyst in the process according to the invention is considerably increased by the presence of bismuth and/or bismuth compounds thereof, which has a positive effect on the reusability of the palladium catalyst.

The amount in which bismuth and/or bismuth compounds thereof are used can be varied within wide limits. For instance, the molar ratio of bismuth or bismuth compound to 3,3-dimethyl-2-hydroxybutyric acid or salts thereof used can be, for example, 1·10⁻⁶ to 1·10⁻¹, preferably 5·10⁻⁶ to 1·10⁻¹ and particularly preferably 2·10⁻⁵ to 2·10⁻². Larger amounts are possible, but uneconomic.

Bismuth can be used, for example, in elemental form and/or in the form of bismuth compounds, e.g. as the oxide, hydroxide, oxide hydrate or salt of a hydrogen acid such as chloride, bromide, iodide, sulphide or as the salt of an inorganic oxygen acid such as nitrate, nitrite, phosphite, phosphate, carbonate, perchlorate or as the salt of an oxygen acid which is derived from a transition metal, or as the salt of an organic acid or as a complex or as an organometallic compound.

It is also possible to introduce bismuth and/or bismuth compounds into the process according to the invention in combination with other metals, semimetals or compounds thereof.

The bismuth (activator) to be used according to the invention can be present in different and also mixed valencies. It is also possible for changes in the valency to occur during the reaction. It is also possible that the bismuth and/or the bismuth compounds are partly or completely converted to other compounds in the reaction medium.

The bismuth and/or the bismuth compounds can be added to the reaction components as a solid, preferably in finely divided form. It is also possible, however, to add the bismuth and/or the bismuth compounds when the palladium catalyst is prepared, or to impregnate the palladium catalyst with bismuth and/or the bismuth compounds. Bismuth and/or the bismuth compounds can also serve as a support material for the palladium.

The concentration of the organic compounds (3,3-dimethyl-2-hydroxybutyric acid and its reaction products) in the reaction solution is preferably selected in such a way that both the 3,3-dimethyl-2-hydroxybutyric acid and the 3,3-dimethyl-2-oxobutyric acid formed are predominantly or preferably completely dissolved under the reaction conditions. The 3,3-dimethyl-2-hydroxybutyric acid can optionally be added to the oxidation mixture (optionally together with base) gradually (continuously or in portions). Useful concentrations of organic compounds have proven to be 5 to 30% by weight. Lower concentrations, for example, are also possible, but less economically viable.

The process according to the invention is preferably carried out in the presence of water and base. The amount of base is preferably such that a total of 1.1 to 8, preferably 1.2 to 6, molar equivalents of base are used per mole of 3,3-dimethyl-2-hydroxybutyric acid to be oxidized. When the 3,3-dimethyl-2-hydroxybutyric acid is used as the alkali metal salt or in the form of an aqueous solution thereof, the amounts of base which are equivalent to the acid and may already be present in the solution have to be taken into account in these considerations. Larger amounts of base are possible in principle, but uneconomic.

The base used is preferably an alkali metal or alkaline earth metal carbonate or hydroxide, a mixture thereof or a corresponding aqueous solution. Particular preference is given to using sodium hydroxide and potassium hydroxide, a mixture thereof or corresponding aqueous solution.

The reaction can be carried out at a pressure of, for example, 0.1 to 50 bar, preferably 0.5 to 5 bar, particularly preferably 0.9 to 1.5 bar and very particularly preferably at ambient pressure.

The conversion is preferably effected between 20 and 150° C.

Advantageously, a temperature is selected within the range from 60° C. to the boiling point of the reaction mixture at the selected reaction pressure.

In a particularly preferred embodiment of the process according to the invention, the lower temperature limit t (lower) is selected in such a way that it obeys the formula

t(lower)=90−n² (° C.)

where

n is the number of equivalents of alkali per mole of 3,3-dimethyl-2-hydroxybutyric acid (DHBA for short) used.

When using 5 mol of NaOH/mole of DHBA, for example, it is possible, for example and with preference, to work within the range from about 65° C. to the boiling point, when using 3 mol of NaOH/mole of DHBA, for example and with preference, to work within the range from about 81° C. to the boiling point, when using 2 mol of NaOH/mole of DHBA, for example and with preference, to work within the range from about 86° C. to the boiling point, and when using 1.5 mol of NaOH/mole of DHBA, for example and with preference, to work within the range from about 88° C. to the boiling point of the reaction mixture.

Very particular preference is given to working within the range from 90° C. to the boiling point, since this temperature range allows high yields in a short time both at low and at high alkali/DHBA molar ratios, and the high temperatures at the same time enable good removal from a reaction technology point of view of the heat of reaction of the exothermically proceeding oxidation.

The sequence in which the palladium catalyst, bismuth and/or bismuth compounds, water, base and 3,3-dimethyl-2-hydroxybutyric acid are combined, may be selected as desired and is uncritical. For instance, palladium catalyst and bismuth and/or bismuth compounds can be added to the mixture or solution of water, base and 3,3-dimethyl-2-hydroxybutyric acid. It is also possible to initially charge the palladium catalyst and bismuth and/or bismuth compounds and add the mixture or solution of water, base and 3,3-dimethyl-2-hydroxybutyric acid. Finally, it is also possible to initially charge the palladium catalyst, a portion of the water-base mixture and also bismuth and/or bismuth compounds, and then add the 3,3-dimethyl-2-hydroxybutyric acid, optionally together with the remaining base or remaining mixture of water and base. It is also possible to add bismuth and/or bismuth compounds to the mixture of the remaining components.

In general, the process according to the invention is carried out in such a way that oxygen or oxygen-containing gases, for example air, are contacted intensively with the reaction mixture which comprises water, base, the palladium catalyst, bismuth and/or bismuth compounds and also the 3,3-dimethyl-2-hydroxybutyric acid.

The catalyst need not be present as a powder suspended in the reaction mixture, but can also be arranged in granular form as a fixed bed which is flowed through by the remaining components.

The progress of the reaction may be followed, for example, via the measurement of the amount of oxygen taken up. The reaction can be terminated when the amount of oxygen required for achieving an optimal selectivity has been taken up. To achieve high selectivities, the reaction is terminated preferably at an oxygen take-up of 0.2 to 0.5 mol of oxygen/mole of 3,3-dimethyl-2-hydroxybutyric acid, particularly preferably after 0.4 mol to 0.5 mol of oxygen/mole of 3,3-dimethyl-2-hydroxybutyric acid. When the intention is to achieve a good yield, the reaction is terminated preferably after take-up of 0.4 to 0.6 mol of oxygen/mole of 3,3-dimethyl-2-hydroxybutyric acid, particularly preferably after take-up of 0.45 to 0.58 mol of oxygen/mole of 3,3-dimethyl-2-hydroxybutyric acid and very particularly preferably after take-up of 0.50 to 0.56 mol of oxygen/mole of 3,3-dimethyl-2-hydroxybutyric acid. Which value is advantageous depends, upon the selected reaction and work-up conditions, the desired product purity, etc., and may be determined in individual cases by preliminary experiments.

The progress of the reaction can also be monitored in other ways, for example by determining the 3,3-dimethyl-2-hydroxybutyric acid consumed, the 3,3-dimethyl-2-oxobutyric acid formed and/or the trimethylacetic acid formed therefrom in a small amount towards the end of the reaction by over-oxidation in a subsequent reaction.

The work-up in the process according to the invention can be effected by customary methods. In general, the palladium catalyst is removed together with undissolved and adsorbed bismuth and/or bismuth compounds, for example by filtering. The solution obtained which contains the 3,3-dimethyl-2-oxobutyric acid predominantly in the form of its salts in high yield can, optionally after partial or complete neutralization of the superstoichiometric base, be used further as such. However, it is also possible to release the 3,3-dimethyl-2-oxobutyric acid from the solution by acidifying with a mineral acid, e.g. hydrochloric, sulphuric or phosphoric acid, and isolate it by known processes, for example by extraction with a sparingly water-soluble organic solvent, e.g. ethers (e.g. diethyl ether, diisopropyl ether) or ketones (e.g. methyl isobutyl ketone). The extraction can also be effected at different pH levels with simultaneous purification (see also U.S. Pat. No. 6,274,766). After distilling off the extractant, a further purification can be effected if required, for example by fractional distillation, optionally under reduced pressure. It is also possible to release the 3,3-dimethyl-2-oxobutyric acid from the alkali metal salt solution initially obtained using a cation exchanger, isolate it by gently evaporating the solution obtained and, if required, further purify it by fractional distillation.

The 3,3-dimethyl-2-oxobutyric acid or salts thereof which can be prepared according to the invention are suitable in particular for preparing agrochemicals, such as metribuzin. It is preferably suitable for preparing 4-amino-6-tert-butyl-3-thio-1,2,4-triazin-5(4H)-one.

The process according to the invention is notable in that oxygen (air) can be used as the oxidizing agent at low pressure, the oxidation proceeds even at low catalyst concentrations in very high yields and the high conversion rates also allow high space-time yields to be realized.

The invention is further illustrated by the following non-limiting examples.

### EXAMPLES

### Example 1

A reaction vessel which is equipped with a stirrer, internal thermometer and gas feed and can be heated using a heating mantle is charged with 1.0 g of activated carbon (medicinal carbon) having a 5% by weight palladium content, 0.024 g of Bi (NO₃)₃.5H₂O, 100 ml of 3N sodium hydroxide solution and 13.2 g (0.1 mol) of 3,3-dimethyl-2-hydroxybutyric acid (DHBA).

After expelling air from the reaction vessel using oxygen, the starting mixture is heated to 95° C. and stirred at this temperature until, after approx. 2 hours, 0.0535 mol of oxygen (approx. 1300 ml at room temperature and ambient pressure) has been taken up. The oxygen feed and the stirrer are then switched off.

After filtering off the catalyst (it can be reused), the filtrate is adjusted to pH 7.5 at room temperature using 20% hydrochloric acid, the amount of filtrate is determined and the 3,3-dimethyl-2-oxobutyric acid content in an aliquot is determined by differential pulse polarography (base electrolyte; acetate buffer solution). The determination was effected against a 3,3-dimethyl-2-oxobutyric acid solution of known content which was used as an internal standard in a repeat measurement on the same sample. The determination gave a 3,3-dimethyl-2-oxobutyric acid yield of 11.7 g (90% of theory).

After acidifying the filtrate to pH 1, repeatedly extracting using ether and vaporizing the ether, approx. 0.7 g (7% of theory) of trimethyl acetic acid could be detected in the extraction residue by gas chromatography in addition to 3,3-dimethyl-2-oxobutyric acid.

### Examples 2-5 (Comparative Examples)

As described in Example 1, 0.1 mol (13.2 g) of 3,3-dimethyl-2-hydroxybutyric acid (DHBA) is oxidized to 3,3-dimethyl-2-oxobutyric acid (DOBA) in sodium hydroxide solution using oxygen under atmospheric pressure over 1 g of noble metal/activated carbon catalyst.

Table 1 reports the type of noble metal/carbon catalyst used, the type and amount of additive, the amount of sodium hydroxide solution used, the oxidation temperature, the amount of oxygen taken up, the time required to take up this amount of oxygen and the DOBA yield achieved which was determined by polarography. For comparison, the values of Example 1 are also listed in Table 1.

TABLE 1

| | | | | | |
|---|---|---|---|---|---|
| Example No. | 1 | 2 | 3 | 4 | 5 |
| Catalyst | 5% Pd/ | 5% Pd/ | 5% Pd/ | 1% Pt/ | 1%Pt/ |
| | act. | act. | act. | act. | act. |
| | carbon. | carbon | carbon | carbon | carbon |
| Additive | Bi(NO₃)₃ | Pb(NO₃)₂ | none | Pb(NO₃)₂ | Pb(NO₃)₂ |
| [mol/mol | 5 · 10⁻⁴ | 1.3 · 10⁻³ | — | 5 · 10⁻³ | 5 · 10⁻³ |
| DHBA] | | | | | |
| Sodium | 3 | 3 | 3 | 4 | 2 |
| hydroxide | | | | | |
| solution | | | | | |
| [mol/mol | | | | | |
| DHBA] | | | | | |
| Temperature | 95 | 95 | 95 | 95 | 70 |
| [° C.] | | | | | |
| O₂ take-up | | | | | |
| [mol/mol | 0.535 | 0.090 | 0.090 | 0.005 | 0.015 |
| DHBA] | | | | | |
| Time | | | | | |
| [hours] | 2 | 8 | 8 | 1 | 1 |
| Stop? | no | almost | almost | yes | yes |
| DOBA yield | 90 | 5 | 5 | <1 (not | <3 (not |
| [% of theory] | | | | worked | worked |
| | | | | up) | up) |

### Examples 6-15

As described in Example 1, 0.1 mol (13.2 g) of 3,3-dimethyl-2-hydroxybutyric acid (DHBA) in 100 ml of 5N sodium hydroxide solution (=5 mol NaOH/mole of DHBA) is oxidized at different temperatures over 1 g of activated carbon (medicinal carbon) having a 5% by weight palladium content in the presence of 5-10⁻⁴ mol of Bi(NO₃)₃/mole of DHBA as an additive at atmospheric pressure until approx. 1.300 ml of oxygen (approx. 0.535 mol of O₂/mole of DHBA) has been taken up. The results with regard to reaction time and 3,3-dimethyl-2-oxobutyric acid (DOBA) yield determined by polarography are compiled in Table 2.

TABLE 2

| | | | | | | | | | | |
|---|---|---|---|---|---|---|---|---|---|---|
| Example | 6 | 7 | 8 | 9 | 10 | 11 | 12 | 13 | 14 | 15 |
| No. | | | | | | | | | | |
| Temp. [° C.] | 65 | 70 | 75 | 80 | 85 | 90 | 95 | 97.5 | 100 | 102.5 |
| Time [h] | 2 | 11 | 5.7 | 3.8 | 2.3 | 1.4 | 1.0 | 2.5 | 7.3 | 19 |
| DOBA | 61 | 73 | 76 | 81 | 77 | 83 | 91 | 92 | 90 | 94 |
| yield | | | | | | | | | | |
| [% of theory] | | | | | | | | | | |

Following the procedure of Example 1, 0.1 mol (13.2 g) of 3,3-dimethyl-2-hydroxybutyric acid (DHBA) in 100 ml of sodium hydroxide solution of different normality is oxidized over 1 g of activated carbon having a 5% by weight palladium content in the presence of 5·10⁻⁴ mol of Bi(NO₃)₃ per mole of DHBA as activator at 95° C. under atmospheric pressure until approx. 1300 ml of oxygen (approx. 0.535 mol of O₂/mole of DHBA) has been taken up. The results with regard to the reaction time and 3,3-dimethyl-2-oxobutyric acid (DOBA) yield determined by polarography are compiled in Table 3:

Example 16 is a comparative example in which no superstoichiometric amounts of base were used.

TABLE 3

| | | | | | | | |
|---|---|---|---|---|---|---|---|
| Example No. | 16 | 17 | 18 | 19 | 20 | 21 | 22 |
| mol of NaOH/ | 1 | 1.5 | 2 | 3 | 4 | 5 | 7.5 |
| mol of DHBA | | | | | | | |
| Reaction time | > > 6^{*)} | 5 | 4.4 | 2 | 1.8 | 1 | 0.7 |
| [h] | | | | | | | |
| DOBA yield | not | 86 | 90 | 87 | 91 | 82 | |
| [% of theory] | determined | | | | | | |

^{*)}After 6 hours, only approx. 2% (0.01 mol of O₂/mole of DHBA) have been taken up and the O₂ take-up has almost come to a stop.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for preparing 3,3-dimethyl-2-oxobutyric acid or salts thereof by oxidizing 3,3-dimethyl-2-hydroxybutyric acid with transition metal catalysis, characterized in that it is carried out in the presence of oxygen, palladium catalyst, bismuth and/or bismuth compounds and base.

2. Process according to claim 1, characterized in that the palladium catalyst used is metallic palladium or palladium applied to a support.

3. Process according to claim 1, characterized in that the palladium catalyst is selected in molar ratio of palladium to 3,3-dimethyl-2-hydroxybutyric acid or salts thereof between 1:5 and 1:20,000.

4. Process according to claim 1, characterized in that activated carbon is used as a support.

5. Process according to claim 1, characterized in that bismuth is used in elemental form and/or a bismuth compound in the form of oxide, hydroxide, oxide hydrate or salt of a hydrogen acid.

6. Process according to claim 1, characterized in that the amount of bismuth and/or bismuth compounds thereof is selected in molar ratio of bismuth and/or bismuth compound to 3,3-dimethyl-2-hydroxybutyric acid or salts thereof between 1·10⁻⁶ to 1·10⁻¹.

7. Process according to claim 1, characterized in that the bismuth and/or the bismuth compounds are used in combination with other metals and/or semimetals or compounds thereof.

8. Process according to claim 1, characterized in that the concentration of 3,3-dimethyl-2-hydroxybutyric acid and its reaction products in the reaction solution is 5 to 30% by weight.

9. Process according to claim 1, characterized in that the amount of base is selected in a total of 1.1 to 8 equivalents of base per mole of 3,3-dimethyl-2-hydroxybutyric acid to be oxidized.

10. Process according to claim 1, characterized in that the base used is an alkali metal or alkaline earth metal carbonate or hydroxide, mixture thereof or corresponding aqueous solution.

11. Process according to claim 1, characterized in that the reaction is carried out at a pressure of 0.1 to 50 bar.

12. Process according to claim 1, characterized in that the reaction is carried out at a temperature of 20 to 150° C.

13. Process according to claim 1, characterized in that the reaction is carried out at a temperature in the range from 60° C. to the boiling point of the reaction mixture at a selected reaction pressure.

14. Process according to claim 1, characterized in that the reaction is terminated at an oxygen take-up of 0.4 to 0.6 mol of oxygen/mole of 3,3-dimethyl-2-hydroxybutyric acid.

15. Process according to claim 1, characterized in that the solution obtained which contains 3,3-dimethyl-2-oxobutyric acid predominantly in the form of its salts is used further after partial or complete neutralization of the superstoichiometric base.

16. Process according to claim 1, characterized in that 3,3-dimethyl-2-oxobutyric acid is obtained by acidifying the solution with a mineral acid, and subsequently extracting.

17. A process for preparing an agrochemicals comprising incorporating 3,3-dimethyl-2-oxobutyric acid which has been obtained by a process according to claim 1 into the agrochemical.

18. The process according to claim 17, characterized in that the agrochemical is metribuzin.
